(19) United States
(12) Patent Application Publication
(10) Pub. No.: US 2024/0228452 A1
(43) Pub. Date: Jul. 11, 2024

(54) Method for Producing alpha-Methylene-Lactones

(71) Applicants: Kirk J Abbey, Garner, NC (US); Jonathan L. KENDALL, Cary, NC (US); Kathleen M. ABBEY, Garner, NC (US)

(72) Inventors: Kirk J Abbey, Garner, NC (US); Jonathan L. KENDALL, Cary, NC (US); Kathleen M. ABBEY, Garner, NC (US)

(21) Appl. No.: 18/037,233

(22) PCT Filed: Nov. 16, 2021
(86) PCT No.: PCT/US2021/059589

§ 371 Date: May 16, 2023

(51) Int. Cl. C07D 307/58

(52) U.S. Cl. CPC C07D 307/58

(57) ABSTRACT

The process consists of a two-step synthesis of α-methylene-γ-butyrolactones from the parent γ-butyrolactones in a single vessel by a process wherein an alkoxide base, a formate ester, and a lactone are reacted using a solvent mixture comprising an aromatic hydrocarbon, and optionally, secondary and/or tertiary alcohols in the first step. By-products that lower overall yield and are difficult to separate from the desired product are eliminated or substantially reduced using a neutralizing agent to convert residual alkoxide to a weak base and/or distillation. In the second stage, a formaldehyde source is introduced to form the final a-methylene lactone. The α-methylene-γ-butyrolactones are useful as monomers for preparing highTg homopolymers or copolymers by various methods for applications with the desirable characteristics of low volatility, increased polymer rigidity, improved optical, and/or thermal stability properties.

### CROSS REFERENCE TO RELATED APPLICATIONS

The present application claims prior under 35 USC § 119 (e) from US Provisional Patent Application Ser. No. 63/205,111 entitled, “Synthesis of alpha-methylene-gamma-butyrolactones” filed on Nov. 16, 2020, the disclosure of which is herein incorporated by reference.

### FIELD OF THE INVENTION

This invention relates to the process of making alpha-methylene lactones, such as those five-member lactones with a single exocyclic unsaturation positioned adjacent to the lactone carbonyl.

### BACKGROUND

Many biologically important sesquiterpenes contain an alpha-methylene-gamma-lactone moiety in their structure. Finding useful analogs to these compounds has generated much synthetic effort. (Select reviews: (a) Gammill, R. B.; Wilson, C. A.; Bryson, T. A.; Synth. Commun. 1975, 5, 245. (b) Grieco, P. A.; Synthesis 1975, 67. (c) Newax, S. S.; Aldrichimica Acta 1977, 10, 64. (d) Hoffmann, H. M. R.; Rabe, J.; Angew. Chem., Int. Ed. Engl. 1985, 24, 94). Further, the simple parent species, alpha-methylene-gamma-butyrolactone (MBL), given the trivial name of tulipalin A, is found in nature at low levels, particularly in white tulips. Numerous publications and patents have appeared directed to MBL and simple ring-alkylated analogs. alpha-Methylene-gamma-valerolactone (MVL), which can be derived from gamma-valerolactone (GVL), along with MBL, have been considered as potential monomers for conversion to homopolymers and copolymers. MBL can be derived from biomass via itaconic acid (Fetizon, M.; Golfier, M.; Louts, J-M; Tetrahedron 1975, 31, 171). GVL can be derived from carbohydrates via hydrogenation of levulinic acid for which many synthetic process studies have been conducted. Both itaconic acid and levulinic acid are listed in the top twelve value added biomass intermediates by the U.S. Department of Energy Office of Energy Efficiency and Renewable Energy. (Werpy, T.; Petersen, G. Top Value Added Chemicals from Biomass. Volume I-Results of Screening for Potential Candidates from Sugars and Synthesis Gas; Department of Energy: Washington DC, 2004).

Despite decades of research on their synthesis, MBL and MVL are available only in research quantities due to the cost of their syntheses. The two most economically promising routes are 1) a direct reaction of a parent lactone with formaldehyde in the presence of base, U.S. Pat. No. 6,232,474 (2001) or 2) a two-stage method to convert a parent lactone into an α-carbonyl lactone salt, followed by reaction with formaldehyde, ((a) Murray, A. W.; Reid, R. G.; Synthesis 1985, 35; (b) Jenkins, S. M.; Wadsworth, H. J.; Bromidge, S.; Orlek, B. S.; Wyman, P. A.; Riley, G. J.; Hawkins, J.; J. Med. Chem. 1992, 35, 2392. Orlek, B. S.; Faulkner, R. E.; U.S. Pat. No. 5,166,357 (1992); and Lee et al., U.S. Pat. Pub. 2015/0073156 A1. Lee et al. U.S. Pub. 2015/0073156 discloses working examples of a two-stage process using THE as a solvent. The suggested other solvents are primary alcohol such as methanol, ethanol, n-butanol; and ethers including tetrahydrofuran (THF), dioxane and aromatic compounds including toluene and xylene; and polar solvents.

A three-stage method is disclosed in U.S. Pat. No. 2,624,723 wherein the initially prepared alpha-formylated lactone is hydrogenated to the alpha-methylol derivative and then pyrolyzed to generate MVL or MBL.

The direct, single step synthesis of MBL and MVL from economical parent lactones and a formaldehyde source under basic conditions is disclosed using a solution phase in U.S. Pat. Nos. 6,232,474, 7,141,682, 7,199,254, 7,205,416; a gas phase in U.S. Pat. Nos. 6,313,318, 7,151,185, 7,161,014, 7,164,033; and in a supercritical phase in U.S. Pat. Nos. 6,649,776, 7,153,981, 7,164,032, 7,166,727, 7,314,942. The highest selectivity and yield reported occurred in a continuous supercritical carbon dioxide phase using a catalyst prepared from rubidium acetate on a high surface area silica. Apparent drawbacks of this process are rapid yield decrease over time due to catalyst bed fouling and use of costly rubidium, despite partial regeneration of this catalyst.

A continuous gas phase reaction with silica-supported metal ion catalysts is disclosed in JP 3,148,134 B2 and U.S. Pat. No. 6,313,318. Notable drawbacks include modest initial conversions of reactants, limited product selectivity, rapid catalyst fouling and low ratio of catalyst on-line time to regeneration time. U.S. Pat. No. 6,232,474 discloses a single-stage reaction of lactones and formaldehyde in the presence of a base in the liquid phase (with or without solvent) over a temperature range of 80-130° C. Total quantitative yields that included by-products were not reported. Example 12 raised the conversion of product obtained in Example 11, absent solvent, from 65% to 78% (excluding by-products) using solid Cs2CO3 base followed by further additions of paraformaldehyde and base in the form of K2CO3, however 22% of unreacted starting lactone remains in the product mixture.

Although the direct methods are seemingly attractive as one-step syntheses, they all result in a crude product that contains an uneconomical level (>10%) of unreacted starting lactone; the presence of unreacted starting lactone limits separation and purification with close boiling points between the starting lactone and products and the thermal instability of the alpha-methylene products.

In contrast, the two-stage processes comprising the initial transformation of a parent lactone into an α-carbonyl lactone salt followed by reaction with formaldehyde holds potential for more complete transformation to an intermediate, easier separation of starting lactone from the intermediate, or both. Of the two-stage reactions, the lactone-formate ester approach appears to be more economical. The molecular weight and cost of formate esters (Murray, A. W.; Reid, R. G.; Synthesis 1985, 35) are lower than oxalate esters (U.S. Pat. No. 6,531,616). However, in the method of Murray supra, the use of sodium hydride in diethyl ether generates hydrogen gas, which must be cautiously vented, and the diethyl ether used in a large amount is flammable, expensive, and susceptible to peroxide formation. In U.S. Pat. No. 6,531,616, the formate route was used in the comparative examples wherein sodium hydride was replaced with sodium ethoxide, and the diethyl ether was replaced with tetrahydrofuran (THF). However, up to 5% starting lactone was present in the product after distillation rendering higher purity levels uneconomical to attain for the reasons mentioned above.

US Patent application 2015/0073156 discloses a two-stage solution process with improved particle filtration time and yield based on controlled agitation rates. The disclosed solvents are primary alcohols and ethers.

The conventional use of THE presents drawbacks due to relative cost and susceptibility to peroxide formation. Although peroxide-forming solvents can be used in industrial processes, great care and expense must be taken to limit their oxidation. Further, the amount of solvent and processing steps involved makes the conventional procedures uneconomical. Therefore, a need still exists for a lower cost, more robust process for the synthesis of MBL, MVL, and related alpha-methylene-gamma-alkyl-gamma-butyrolactones.

### SUMMARY OF THE INVENTION

The present invention provides an improved two-stage method for producing alpha-methylene lactone from formate esters wherein the process comprises a first stage and a second stage, wherein during the first stage a formate ester is reacted with a lactone using a metal alkoxide base to form an enolate salt. The first stage reaction is carried out in the presence of a solvent mixture detailed hereinbelow which comprises an aromatic hydrocarbon and optionally secondary and/or tertiary alcohols. Further embodiments increase product yields by conducting the second stage reaction of the enolate salt with formaldehyde in the substantial absence of primary alcohol which is removed prior to the second stage. Further embodiments increase product yields by neutralizing the metal alkoxide by a neutralizing agent which results in weakly basic conditions for further reaction of the enolate intermediate in the second stage. It has been found that primary alcohols added as solvent or present as a by-product of reacted metal alkoxides and/or formate esters reduces the conversion of the lactone whereas employing a particular solvent mixture according to the invention overcomes this drawback. In another aspect of the invention, the process includes the substantial removal of primary alcohol(s) prior to conducting the second stage reaction of with formaldehyde resulting in yet higher conversion of lactone to product on a theory that the extent of base-induced side reactions is reduced. In another aspect of the invention the process includes neutralizing unreacted metal alkoxide from the first stage using a neutralizing agent that dissociates into a weak base carried into the second stage reaction of the alpha-formyl lactone salt.

### BRIEF DESCRIPTION OF FIGURES

FIG. 1. depicts one embodiment of the process in a first stage by reacting a formate ester A with a lactone B and metal alkoxide C in solvent to form crude mixture containing alpha-formyl lactone salt D, alcohols E and formate esters F. R₁ is a lower alkyl, linear or branched, of 1-6 carbons, R₂ is hydrogen or a lower alkyl, linear or branched, of 1-6 carbons, R₃ is a lower alkyl, linear or branched, of 1-6 carbons, and R₄ is an alkyl group introduced with the alcoholic solvent component.

FIG. 2 depicts the second stage reaction of alpha-formyl lactone salt D with a formaldehyde source H to form product alpha-methylene-gamma-butyrolactone I and a formate salt J.

FIG. 3 depicts a side reaction of alpha-methylene-gamma-butyrolactone I with a strong base C′ (alkali alkoxide where all alcohols exchange) and alcohols K, where Rₙ=R₁, R₃, or R₄ defined above, to give oxa-Michael by-products S and other by-products T and P. Agent, Q, neutralizes the alkoxide bases, C′, and creates weak base Q′ and the alkali metal ion M⁺ is associated with X⁻, the counterion of the neutralizing agent, Q, greatly diminishing or total eliminating the by-products S, T, and P.

### DETAILED DESCRIPTION OF THE INVENTION

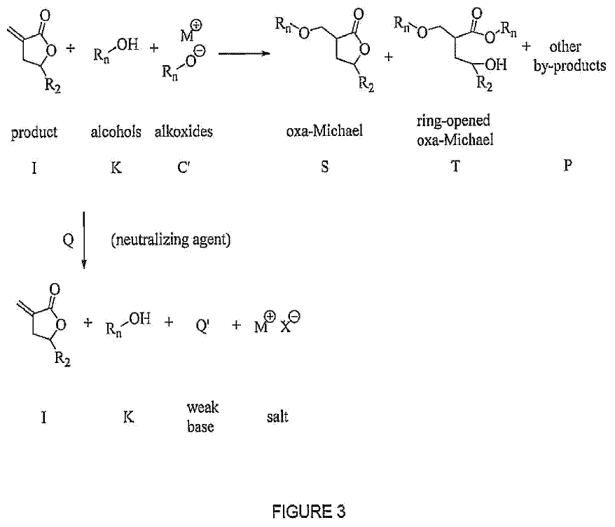

The invention described herein comprises an improved process to synthesize alpha-methylene lactones such as alpha-methylene-gamma-butyrolactone and alpha-methylene-gamma-alkyl-gamma-butyrolactones (hereinafter collectively referred to as alpha-methylene lactones and product) in a two-stage solution reaction in which a solvent mixture comprising aromatic hydrocarbon and secondary and/or tertiary alcohol. It has been found that secondary and/or tertiary alcohols in the substantial absence of primary alcohols combined with aromatic hydrocarbon facilitates improved conversion to enolate salt when an alkali alkoxide is used as the base. In another embodiment, using the defined solvent mixture a further aspect of the invention is controlling, i.e., reducing or eliminating primary alcohols added or generated in the Stage 1 reaction prior to the second stage which provides an unexpected reduction of side reactions. This further aspect also reduces the incidence of oxa-Michael type by-product S (FIG. 3). In a further preferred aspect of the invention, a step of neutralizing excess strong alkoxide base remaining from the first stage is included which also has been found to reduce second stage side reactions and further conversion of oxa-Michael type by-products. In yet a further preferred aspect, the neutralization of alkoxide is done by employing a neutralizing agent which itself converts to a weak base.

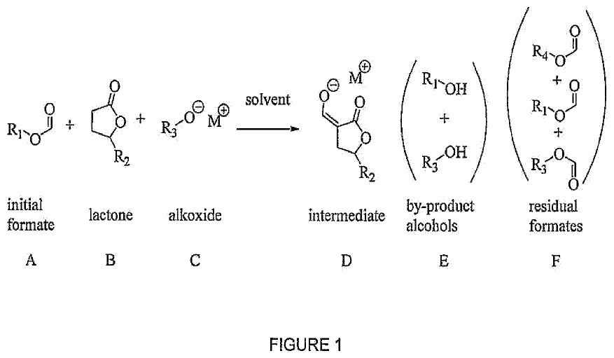

Referring to FIG. 1, which illustrates an embodiment of a first stage, formate ester A is reacted with gamma-butyrolactone B using a metal alkoxide C in a solvent mixture defined herein in an equilibrium favoring the product enolate anion. By-product alcohols E and formates F are generated from the starting material by conversion of metal alkoxide, and secondary and/or tertiary alcohol. In reference to formate ester A, R₁ is primary alkyl or secondary alkyl; referring to lactone B, R₂ is H or (linear or branched) alkyl of between 1 and 6 carbons; referring to metal alkoxide C, R₃ is lower alkyl, linear or branched; referring to one formate in the group of F where R₄ is a secondary and/or tertiary alkyl group, or both, containing from 3 to 6 carbons; referring to metal alkoxide C, M⁺ is an alkali metal cation such as sodium, potassium and lithium. In one embodiment A is a formate ester, B is a gamma-butyrolactone, C is and alkoxide base, D is the alpha-formyl salt of the lactone B, E consists of alcohols generated in the reaction and F consists of a mixture of formate esters created by base induced transesterification from alcohols present.

In a further aspect, as the first stage reaction proceeds, the mass of alcohols increases and thus the make-up of the solvent mixture changes as the reaction proceeds beginning with the solvent mixture charged to the reactor, namely, a mixture comprising aromatic hydrocarbon and secondary and/or tertiary alcohol, and further including additional primary or secondary alcohol by-products depending on the metal alkoxide(s) employed.

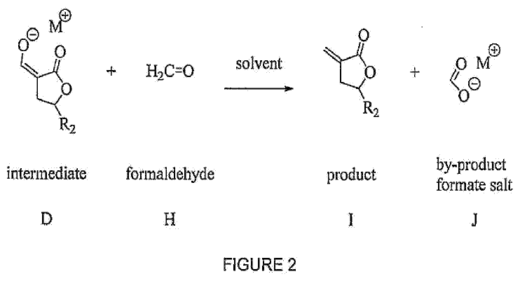

Referring to FIG. 2, which illustrates a second stage embodiment of the reaction commenced in stage 1 of FIG. 1, after substantially eliminating primary alcohols in a step not shown, the solvent mixture of the second stage comprises an aromatic hydrocarbon, minimal residual primary alcohol, and secondary, and/or tertiary alcohols of the solvent mixture; H is formaldehyde; I is the desired product alpha-methylene-gamma-butyrolactone and J is a formate salt.

Referring to FIG. 3, which illustrates conversion of product to by-products in the presence of strong alkoxide base, the alpha-methylene-gamma-butyrolactone I reacts with alcohols (RₙOH where Rₙ=R₁, R₃, and R₄ previously defined to give oxa-Michael by-products, S, which further undergo ring-opening reactions with alcohols to form species T, and other by-products P not shown. Treating the alkoxide base with an agent for neutralization, Q, forms weak base, Q′, and alkali metal salts associated with the counterion, X⁻, from Q and, in accordance with a preferred aspect, significantly reduces or completely eliminates the generation of side products.

Suitable aromatic hydrocarbons used herein include benzene, toluene, isomers of xylene, or ethylbenzene, or a mixture of aromatic compounds.

Primary alcohols are defined to be hydrocarbyl compounds with at least two hydrogens bonded to the carbon bound to the OH group, i.e., methanol, ethanol, 1-propanol, 2-methyl-1-propanol (isobutanol). Primary alkyl groups are defined to mean hydrocarbyl compounds with at least two hydrogens on first carbon bonding the group such as methyl-, ethyl-, n-propyl-, 2-methylpropyl (isobutyl), and the like. Secondary alcohols include isopropanol, sec-butanol, 3-methyl-2-butanol, hexan-2-ol, hexan-3-ol, 3-methylpentan-2-ol, 4-methylpentan-2-ol, and 2-methylpentan-3-ol. Tertiary alcohols include tert-butanol or tert-pentanol (2-methyl-2-butanol), 3-methylpentan-3-ol, 2,3-dimethylbutan-2-ol, and 2-methylpentan-2-ol. It is preferred to have between 1 and 80 weight percent combined secondary and tertiary alcohols. The most preferred content of combined secondary and tertiary alcohols is 5 to 60 weight percent of the total solvent.

Suitable alkyl formate esters used in the synthesis in accordance with the invention include methyl formate, ethyl formate, n-propyl formate, isopropyl formate, sec-butyl formate, n-butyl formate, sec-butyl formate, tert-butyl formate or higher alkyl formates. As shown in FIG. 1, upon reaction with the lactone, the formate esters produce alcohols which thus become co-solvents in the reaction. Therefore, in a further embodiment of the invention, formate esters of non-primary alcohols such as isopropyl formate and sec-butyl formate are desirable starting materials. The molar amount of formate relative to the lactone ranges from 1.01:1.00 to 2.00:1.00. Alkyl formate esters are produced commercially by reaction of an alcohol, such as methanol with gaseous carbon monoxide in the presence of a sodium methoxide catalyst. Higher alkyl formates are known and prepared similarly or by transesterification with methyl formate. Commercial suppliers include Eastman Chemical and Parchem.

Suitable lactones for use in the invention besides gamma-butyrolactone, where R₂ is hydrogen, include γ-butyrolactones containing alkyl substituents, R₂, in the gamma-position, wherein R₂ is selected from linear or branched alkyl groups having from 1 to 6 carbons.

Suitable formaldehyde sources include, but not limited to, formaldehyde, hemi-formals of secondary alcohols, and paraformaldehyde.

Alkali metal bases suitable for the first stage reaction to generate the alpha-formyl lactone salt are sodium methoxide, sodium ethoxide, sodium isopropoxide, potassium isopropoxide, sodium tert-butoxide, potassium tert-butoxide, sodium tert-pentoxide, lithium methoxide, lithium ethoxide, lithium propoxide, and lithium t-butoxide, and mixtures thereof. Alkali alkoxides such as those derived from secondary or tertiary alcohols are particularly desirable starting materials which form corresponding secondary or tertiary alcohols forming the solvent mixture according to the invention. The use of above-stoichiometric levels of base improves the conversion of starting lactone. A suitable mole ratio of base to lactone used in the stage one reaction is from 1.00-1.40, and particularly from 1.05-1.35. Alkali metal bases are commercially available from Albemarle Corporation.

The order of addition of reagents can be accomplished in at least two manners: 1) slow addition of a mixture of formate ester and lactone to an agitated dispersion or solution of alkali alkoxide in solvent, or 2) slow addition of solid alkoxide or a solution or dispersion of alkali alkoxide in solvent to an agitated mixture of formate ester and lactone.

The first stage is performed at temperatures below 60° C. with the combination of reagents being initially accomplished starting at ambient temperature of about 20 to 25° C. A slight exotherm of <5° C. is noted for reactions performed at ≤6 liters scale. The reaction exotherm was lower when higher alcoholic co-solvent content was used. Particularly, reaction temperatures below about 40° C. are desirable.

On addition of the mixture of lactone and formate ester solution to the reaction vessel foaming may occur early in the addition and is believed to arise from carbon monoxide gas from base-induced decomposition of the formate esters, therefore excess molar amounts of formate ester are desired to compensate.

Without being bound by any theory, the complete solubility of the alkoxide in the reaction medium may not be a limiting condition for conversion as dissolution of alkoxide proceeds by the disappearance of the soluble portion as it reacts and by the alcohols acting as co-solvent as formed by the reaction of the formate ester and alkoxides.

### Post-First Stage Treatments

Post First Stage treatments have been found to be beneficial in some embodiments to maximize the overall product yield, reduce by-product formation and facilitate product purification at the conclusion of the second stage. These further aspects include conversion of starting lactone to product, non-tertiary alcoholic solvents, and handling of excess alkoxide base.

The invention herein provides for control of unreacted lactone in the crude product mixture of less than about five mole percent relative to the starting lactone, particularly maintaining the lactone level to less than three mole percent, and most particularly less than one mole percent which will facilitate isolation of the alpha-methylene lactone product.

It has been found, according to some embodiments, material loss is lowered by the optional step of reduction of basicity in the second stage reaction, thereby reducing the reaction of remaining starting lactone and formaldehyde. Maintaining weak basicity is believed to generate more desired unsaturated lactone product. In the second stage reaction, the unreacted starting lactone can react directly with formaldehyde if a strong base is still present. While this will generate more desired unsaturated lactone product, it also generates an undesired equivalent amount of water which subsequently reacts with formaldehyde to generate methanol through the Cannizzaro reaction. Any appreciable primary alcohol present with strong base will react to form the oxa-Michael product and further side products which are difficult to separate from the monomer in purification.

Isolation of the alpha-formyl lactone salt by filtration may be employed to remove unreacted starting lactone. However, direct isolation of the salt after the first reaction may not give the desired highest yield because of the appreciable solubility of alkali alpha-formyl lactone salt when aromatic hydrocarbon/secondary or tertiary alcohol mixtures that also contain primary or secondary by-product alcohols derived from the formate ester and alkoxide reagents are used. alpha-Formyl-gamma-valerolactone sodium salt is soluble in 80% toluene/20% tert-butanol by weight at 2.2 weight percent at 20° C., but less than 0.02 weight percent in 100% toluene. The salt's solubility is higher if primary or secondary alcohol by-products are present.

It has been found that the presence of non-tertiary alcohols, if carried into the second stage reaction at more than fifty mole percent based on starting moles of lactone, can react with the product alpha-methylene lactones in a base promoted oxa-Michael reaction to generate more than five mole percent oxa-Michael by-product. Like unreacted starting lactone, the oxa-Michael by-products are difficult to separate from the monomer product and complicate the latter's purification. It has been found that the oxa-Michael products are susceptible to ring-opening reactions. The susceptibility of MVL to oxa-Michael reaction decreases in the order methanol>>ethanol>isopropanol>tert-butanol≅0. In accordance with a preferred aspect, removal of primary and, to a lesser extent, secondary alcohols under vacuum after the first stage reaction reduces the source of side reactions and at the same time decreases the alpha-formylate lactone salt solubility if filtration is a selected separation method. No filtration operation is preferred as it can require excessive time and further solvent consumption.

An alternate alcohol removal process when methyl formate and/or alkali methoxide is used for the first stage reaction is the adsorption of methanol by activated 4 Å molecular sieves in a Soxhlet-style process. This is best performed under reduced pressure and at less than 40° C. from the first stage reaction mixture. Again, this process is not preferred as it adds considerable time and cost.

Unreacted alkoxide base of the first stage is kept at a sufficiently low level, preferably below 5 mole percent relative to the starting lactone, more preferably below 3 mole percent, and most preferably below 1 mole percent by use of neutralization agents such as trialkylammonium salts of protic acids such as hydrogen chloride, acetic acid, and formic acid. Inorganic neutralizing agents such as carbon dioxide or alkali bicarbonates are also useful. An agent for neutralization having a pKa between 9.0 and 13.5 are useful, preferably between 9.0 and 12.0, because they form weak bases to control the basicity in stage two.

Although the two stages of the process of the invention may be undertaken in separate vessels in a semi-batch mode, a one-pot synthesis is preferred. After the reaction in the first stage, alkoxide base is neutralized to a sufficiently low level, or primary alcohols removed at the completion, or both.

### Second Stage Reaction (FIG. 2)

After achieving the necessary low levels of unreacted starting lactone and unreacted alkoxide or any non-tertiary alcohols, or both, an anaerobic free radical inhibitor, or mixture thereof, is added to the alkali alpha-formyl lactone salt and solvent mixture before beginning the addition of the formaldehyde source. Suitable inhibitors include (2,2,6,6-tetramethylpiperidin-1-yl)oxyl (TEMPO), 4-hydroxy-2,2,6,6-tetramethylpiperidin-1-oxyl (TEMPOL), p-phenylenediamines, phenothiazine, hydroxylamines like hydroxypropylhydroxyamine (HPHA) and diethylhydroxylamine (DEHA), quinones, and quinone methides and can be used at 50 to 1,000 ppm.

The addition of a formaldehyde source, such as paraformaldehyde with a purity of 92-97% formaldehyde, is then begun at between 20 to 35° C. either as a single addition or in stepwise incremental additions. The final amount of added formaldehyde is desired to be from one-and-a-half to two-and-one-half formaldehyde equivalents, more preferred one-and-three-quarters to two-and-one-quarter equivalents, based on the initial moles of lactone used.

The conversion of alkali alpha-formyl-lactone salt to alpha-methylene lactone ensues exothermically with the further additions of formaldehyde being timed to maintain a reaction temperature of less than 70° C., more preferably less than 50° C. The second stage reaction is then run to completion by holding this upper temperature for about three hours.

The product may be isolated by various processes common to the art. Also, in accordance with one aspect of the invention, the product can be produced in “one pot”, that is, without removing the crude intermediate from the vessel used in the first stage before conducting the second stage. The reaction mixture may be filtered to remove insoluble solids such as unreacted paraformaldehyde or alkali formate salts. The latter species may also be separated by addition of water to extract it and other water-soluble salts. The amount of water used must be low enough to not extract alpha-methylene-gamma-butyrolactone products. The resultant organic layer can be treated with a drying agent, such as anhydrous magnesium sulfate, filtered, and solvents removed by distillation, preferably at reduced pressure. The product can then be purified by distillation at reduced pressure.

### EXAMPLES

The improvements of the process according to the invention are illustrated by the following examples 1-5 as contrast to comparative examples 1-4.

### General Preparations.

All reagents and solvents were purchased from commercial sources unless otherwise specified. Pure alpha-methylene-gamma-valerolactone for reference purposes was synthesized according to the oxalate method disclosed in Examples 7 and 8 in U.S. Pat. No. 6,531,616. Gas chromatographic (GC) analyses were performed on an Agilent 6890 or 8860. GC response factors of starting materials and products were measured from solutions of known concentrations and were used to determine the concentration of unknown samples to calculate conversion and yield relative to the mole amounts of starting lactone. It was found that the response factors for product and starting materials were not equal as presumed in U.S. Pat. No. 6,232,474. Yield determinations are presented below using both the correct response factors and according to the method reported in '474 for comparison purposes.

Isopropyl formate was prepared (adapted from sec-butyl formate synthesis in U.S. Pat. No. 10,570,081) by distilling the ester away from a mixture of isopropanol and formic acid (≥85% active). The distillate was dried over anhydrous potassium carbonate to remove any water and formic acid that co-distilled. The formate ester was redistilled from phosphorus pentoxide to reduce the amount of isopropanol. The product isopropyl formate contained minor amounts of isopropanol.

sec-Butyl formate was prepared by azeotropic distillation of water and cyclohexane from a mixture of formic acid and 2-butanol using a Dean-Stark trap. After water removal was complete, the cyclohexane was distilled off. The product was then distilled from potassium carbonate and stored over 3 Å molecular sieves.

### Comparative Example 1 (Supercritical Carbon Dioxide Direct Reaction)

During our investigations of the supercritical carbon dioxide direct reaction, the results approached the reported selectivity, but we discovered that the recovery of product, i.e. yield, was substantially lower than theoretical. For example, in a test reaction, while MVL represented about 80% of the recovered lactones, only 64% of the lactone feed was recovered, giving an overall yield of 51%. It was found that gamma-valerolactone even without a formaldehyde source could not be recovered from the continuous supercritical process in better than about 85% of that fed to the reactor as compared to a non-reactive internal standard diphenyl ether. With this poor recovery based on starting lactone and the drop off in conversion in just a few hours of operation and the high levels of unreacted starting lactone in the product mixture, this supercritical carbon dioxide process's yield overall fell into the same range as the solution processes. Because of the elaborate high-pressure equipment needed for the supercritical operation versus atmospheric solution processes, this route became unattractive.

### Comparative Example 2 (Direct Reaction)

While direct base-induced reactions would be assumed to have an economic advantage versus two-stage reactions, disadvantages were observed. At elevated temperatures (80-130° C.) the incidence of reactor fouling was severe and a dark red to black solid crude mix resulted. All bases tested, except Cs₂CO₃, resulted in the base being a stoichiometric reagent and not a catalytic reagent as the reaction scheme would suggest. Further, cesium carbonate is expensive and, as noted in the discussion of U.S. Pat. No. 6,232,474 above, showed only modest catalytic action.

### Comparative Example 3 (Two-Stage Process)

The first stage process of S. M. Jenkins et al., J. Med. Chem. 1992, 35, 2392-2406, was followed except that the diethyl ether solvent was replaced by anhydrous toluene. One liter of azeotropically dried toluene was placed in a 3-L, 3-necked-round-bottomed flask. Sodium hydride (1.6 mol) was transferred to the toluene in the round-bottomed flask in a glove bag under a nitrogen atmosphere. After configuring the reactor assembly with an overhead stirrer, an equalizing pressure addition funnel, and nitrogen inlets and exits, about 6 mL of absolute ethanol was added slowly from the addition funnel while initially at ambient temperature. The resulting temperature was 25° C. Distilled GVL (0.9829 mol) and ethyl formate (1.006 mol) were mixed and placed in the equalizing pressure addition funnel. After about one-fourth of the reactants were added over twenty minutes without any temperature change and only a slow rate of hydrogen evolution, the reaction quickened until rapid gas evolution and a sharp temperature rise to 43° C. occurred before the reaction temperature was controlled with a water bath. The feed was halted during this excursion for about ten minutes until the gas evolution slowed. Once the feed was resumed, the reaction was controlled at about 35° C. After approximately one-half of the reactant feed had been added, the precipitating mass could not be adequately stirred as an immobile outer annulus of precipitate prevented reactants from being mixed and soon the entire surface of the reaction mixture became immobile, albeit the stirring paddle still turned, and was later discovered to have created a cavity within the precipitated mass. The volume of the reaction mixture also was observed to steadily expand at this stage from trapped hydrogen gas. The reaction had to be aborted at this stage.

### Comparative Example 4 (Two-Stage Process—First Stage)

This reaction also used azeotropically dried toluene as solvent but used solid sodium methoxide and methyl formate. In a 3-neck, 500 mL round-bottomed flask, 180 mL of azeotropically dried toluene was combined with 0.203 mol of sodium methoxide under a nitrogen atmosphere in a glove bag. A mixture of distilled GVL (0.190 mol), methyl formate (0.212 mol), and diphenyl ether (9.45 mmol, used as a non-reactive reference for subsequent analysis by GC) was added to an equalizing pressure addition funnel. The toluene/sodium methoxide mix was raised to 38° C. before feeding the reactants over 46 minutes. During the addition, no exotherm was observed nor did the precipitation retard the stirring. The reaction was continued for 80 minutes during which some thickening of the precipitated mass was noted. Samples for GC analysis were taken 36 minutes and 81 minutes after the addition. About 43% and 33% unreacted GVL were still present after 36 minutes and 81 minutes, respectively.

### Comparative Example 4 (Second Stage)

To the product of the first stage of Comparative Example 4 at 81 minutes, paraformaldehyde, 0.384 mol active formaldehyde, was added all at once without filtration nor with neutralization of the excess alkoxide base. Within five minutes, the reaction temperature had risen from 38 to 61° C. and had begun to turn a reddish-orange tint. The temperature then slowly dropped, but the color intensified to a dark red. The reaction mixture had dropped back to 38° C. after 35 minutes. A sample for GC analysis was taken after 60 minutes from the paraformaldehyde introduction. The analysis of the second stage reaction showed a further decrease in starting lactone GVL (with 30% remaining), but most of the MVL product generated had reacted further with methanol to give the oxa-Michael adduct and ring-opened oxa-Michael adducts clearly showing the negative impact of residual primary alcohol on the MVL product yield when a strong base is still present during the reaction between lactone salt and formaldehyde.

### Example 1 (2-Stage Process in a Single Vessel without Removal of Intermediate)

First stage: inside a nitrogen-filled glove bag, a 500 mL, three-necked round-bottom flask was fitted with a mechanical stir rod and paddle. Into the flask was placed 115 mL of 80% tert-butanol/20% toluene which had been dried over 3 Å molecular sieves, and 134.6 mmol of sodium methoxide. The flask was sealed with rubber septa, removed from the glove bag, and placed under a nitrogen flow. A thermocouple and a 250 mL addition funnel were added, and into the funnel was placed 128.2 mmol of GVL, 156.7 mmol of methyl formate, and 4.9 mmol of diphenyl ether (used as a non-reactive GC internal standard).

The flask was stirred and warmed to 28° C. The lactone/formate ester mixture was added over the course of 15 minutes. Bubbles formed on the reaction mixture surface during and after the addition. After about one hour, the mixture was opaque. An additional 22.2 mmol of methyl formate was added dropwise. After 1.5 hours, the mixture began to thicken, but continued to stir well. The reaction was heated at 30° C. for an additional 3 hours. The solvent was then removed under vacuum overnight, to a final pressure of 0.17 torr.

### Example 1 (Second Stage)

The dried solid of Example 1 (first stage) was redispersed with 142 mL of a 25% tert-butanol/75% toluene solvent mixture. The beige suspension was stirred, heated, and treated with 39 mg of phenothiazine. When the flask reached 40° C., paraformaldehyde (263 mmol of active formaldehyde) was added. The reaction exhibited an exotherm to 46° C. and then was maintained at 40° C. for a total reaction time of 3 hours.

The reaction mixture was filtered at 25 psi (Whatman 4). The filter cake was twice redispersed with 100 mL of toluene and filtered. The filtrates were combined and reduced in volume on a rotary evaporator at 33° C. to a pressure of 6.45 torr. A colorless liquid remained. GC analysis of the liquid showed the yield, on a mole basis from the starting lactone, was 3.09% GVL, 50.2% MVL, 1.2% oxa-Michael addition by-product, and 0.3% ring-opened by-product. Other higher boiling by-products were also observed.

For comparison with results reported in U.S. Pat. No. 6,232,474 ('474) as % of MVL product relative to starting lactone, excluding by-products, and assuming equal GC response factors, MVL was 94.2%. Example 1 illustrates a substantial increase in net product obtained, overcoming the appreciable conversion of starting lactone to oxa-Michael addition by-product which has heretofore not been demonstrated.

### Example 2 (First Stage)

To a reaction flask configured according to Example 1, a 200 mL of a mixture of 20% tert-butanol/80% toluene, dried over 3 Å molecular sieves, 204 mmol of solid sodium methoxide was added while enclosed in a nitrogen filled glove bag. GVL (197.9 mmol), methyl formate (228.3 mmol), and diphenyl ether (9.9 mmol) were charged to an addition funnel. After the reactor contents reached 30° C., the GVL mixture was added as in the preceding example. The mixture produced an exotherm of 3° C. Precipitation began after 6 minutes, but never restricted the agitation. An additional 26.6 mmol of methyl formate was added at 27 minutes reaction time.

After heating for a total of 4 hours, the temperature was reduced and the flask placed under vacuum. The volatiles were removed down to 10 torr at 27° C. to leave a paste, which was diluted with 100 mL of toluene and poured into a pressure filter. The flask was rinsed with an additional 50 mL of toluene and poured into the pressure filter. The mixture was filtered at 40 psi. The filter cake was redispersed twice with 100 mL of toluene each and refiltered. The sodium alpha-formyl-GVL salt was dried in the pressure filter down to 3 torr over 2 hours.

### Example 2 (Second Stage)

The dried filter cake (sodium alpha-formyl-GVL salt) was returned to the reaction flask and redispersed in 200 mL Of 20% tert-butanol/80% toluene. The mixture was heated in a water bath to 40° C. Paraformaldehyde (392.1 mmol active formaldehyde) was added and the mixture experienced an exothermic response to a temperature of 48° C. The reaction mixture was inhibited with 44 mg of phenothiazine and maintained at 40° C. for a total of 162 minutes, at which time it was poured into a pressure filter assembly fitted with a Whatman 4 cellulosic filter paper.

After filtration at 25 psi, 100 mL of toluene was added to the filter cake, which was redispersed and filtered again. A second toluene rinse was likewise performed. The filtrates were combined, and the volatiles were removed on a rotary evaporator under vacuum (4 torr) in a heated water bath (31° C.). GC analysis of the clear liquid showed the yield, on a mole basis from the starting lactone, was 0.1% GVL, 51.7% MVL, 0.1% oxa-Michael addition by-product, and 0.2% ring-opened by-product. Other higher boiling by-products were also observed. Following the calculation method of '474, the product relative to starting lactone was 99.8%. Example 2 illustrates a further increase in net product obtained, overcoming the appreciable conversion of starting lactone to oxa-Michael addition by-product which has heretofore not been demonstrated.

### Example 3 (One Pot Method with Neutralization)

First Stage: A 500 mL, three-necked round-bottom flask was fit with a mechanical stirrer, addition funnel, nitrogen inlet, nitrogen outlet, and thermocouple. A 92 mL solvent mixture of 39% tert-butanol/61% toluene and 101.6 mmol of sodium tert-pentoxide were added to the flask, which was then stirred. A solution of 84.8 mmol of GVL and 102.0 mmol sec-butyl formate, and 0.5 mL sec-butanol was placed in the addition funnel. The lactone/formate ester mixture was added to the reaction flask, which was initially 19° C., over ten minutes. An exotherm of 2° C. and gas bubbles were observed during the addition. Another 69.7 mmol of sec-butyl formate was added to the reaction flask. An additional 4.5 mL of toluene was used to rinse any residual ester from the addition funnel into the reaction flask. The reaction was then heated with a water bath from 30° C. and maintained at about 42° C. for six hours. The mixture was opaque, but not thick.

The reaction was cooled and 35.6 g (34.5%) of the solvent mixture was removed under vacuum. Toluene (107 mL) was added to adjust the concentration of the reaction mixture, and 21.1 mmol of triethylammonium hydrochloride was used to neutralize unreacted alkoxide base and release triethylamine resulting in mildly basic pH.

### Example 3 (Second Stage)

Phenothiazine (31 mg) inhibitor and paraformaldehyde (175.4 mmol of active formaldehyde) were added to the alpha-formyl-gamma-valerolactone salt of Example 3 (first stage), and the reaction mixture was heated to 27° C. for 20 hours. A peak exotherm of 34° C. was observed more than an hour past the paraformaldehyde addition. Water was added until there was a clear upper layer (11.219 g total) and the two-phase liquid was transferred to a separatory funnel. The water layer was removed, and the organic layer was dried over MgSO4, filtered, and reduced in volume on a rotary evaporator at 2.9 torr and 30° C. GC analysis of the resulting liquid showed the yield, on a mole basis from the starting lactone, was 1.1% GVL, 74.6% MVL, and some higher boiling species. No oxa-Michael products were present. Following the calculation method of '474, product relative to starting material was 98.5%. Example 3 illustrates a further increase in net product obtained, eliminating conversion of starting lactone to ox-Michael addition by-product which has heretofore not been demonstrated.

### Example 4 (One Pot Method, Neutralization, and Liquid Separation in Stage 2)

First Stage: A reactor assembly of Example 3 was flushed with nitrogen and charged with 102 mL of toluene and 117.8 mmol of sodium tert-pentoxide. Isopropanol (203 mmol) was added to convert the base to sodium isopropoxide and tert-pentyl alcohol. The temperature rose from 20° C. to 30° C., before falling back to 20° C. A solution of 94.3 mmol GVL, 113.5 mmol isopropyl formate, and 8.8 mmol of isopropanol was added to the reaction flask over 20 minutes, during which an exotherm of 3° C. was observed. Another 75.8 mmol of isopropyl formate and 5.8 mmol of isopropanol were added. The reaction mixture became very thick, and 43 mL of toluene was added to improve mixing. The reaction was heated at 39° C. for 2.5 hours and then stirred at room temperature overnight.

Unreacted formate ester and isopropanol produced in the reaction were removed by subjecting the reaction mixture to vacuum (22 torr) until 14.6 g were collected in the cold trap. Toluene (24.8 mL) was added to the reaction mixture to adjust the formate salt concentration. A solution of 28.5 mmol of neutralizing agent triethylammonium acetate in toluene was added to neutralize unreacted alkoxide. An additional 24.9 mmol of triethylamine was added to ensure the formate salt remained in mildly basic conditions.

### Example 4 (Second Stage)

To the alpha-formyl-gamma-valerolactone sodium salt mixture of Example 4 (first stage) was added 62 mg of phenothiazine and 192.6 mmol active formaldehyde as paraformaldehyde. The mixture was heated with a water bath. The reaction was held at 40 to 45° C. for 4 hours, at which time water was added until there was a clear upper liquid layer (14.525 g added). The two-phase liquid was transferred to a separatory funnel, shaken, and allowed to separate. The aqueous layer was extracted with 25 mL of toluene. The toluene layers were combined, dried over MgSO₄, filtered, and reduced in volume on a rotary evaporator at 3.7 torr and 33.2° C. GC analysis of the resulting liquid showed the yield, on a mole basis from the starting lactone, was 2.4% GVL, 53.5% MVL, and 1.6% oxa-Michael by-product. Other higher boiling by-products were also observed. Following the calculation method of '474, product relative to starting material was 95.7%.

### Example 5 (First Stage)

A 500 mL, three-necked round-bottom flask was fit with a mechanical stirrer, reflux condenser, nitrogen inlet/outlet, and thermocouple. Into the flask was placed 120 mL of a solvent mixture comprising 41% tert-butanol, 56% toluene, 1% sec-butanol, 0.4% isopropanol, and 0.3% methanol that was recycled from previous monomer syntheses. The solvent was heated to boiling (81° C.) and stirred at 180 rpm. A suspension of 14 mmol of FeCl₃ in toluene was added to the reaction flask. Over the course of 30 minutes, 193 mmol of sodium metal, freshly cut into eight pieces in a glovebag under a nitrogen atmosphere and stored briefly in toluene, was added to the reaction flask. The reaction was heated at reflux for an additional 2.6 hours. The reflux temperature slowly dropped to 78° C. as a thick suspension developed. The reaction was cooled to 18° C.

The reflux condenser was replaced with a 250 mL addition funnel. A solution of 160.6 mmol GBL, 183.0 mmol sec-butyl formate, and 18 mL of sec-butanol was placed in the addition funnel. The lactone/formate ester was added over the course of 12 minutes. The temperature in the flask rose to 24° C. during the addition. Another 130.8 mmol sec-butyl formate and 13 mL sec-butanol were added to the reaction flask via the addition funnel, which was then rinsed with 24 mL of toluene. The suspension was heated to 37-41° C. for 6 hours before cooling to 20° C. Unreacted alkoxide base was neutralized with 41.0 mmol triethylammonium hydrochloride which converted to triethylamine and rendered the reaction mixture mildly basic. The concentration of alpha-formyl-gamma-butyrolactone salt was adjusted with 87 mL of toluene.

### Example 5 (Second Stage)

To the alpha-formyl-gamma-butyrolactone salt mixture Example 5 (first stage) was added 43 mg of phenothiazine inhibitor and paraformaldehyde (305.7 mmol of active formaldehyde). The reaction mixture was heated at 25-30° C. for 17 hours. Water was added until a clear upper layer formed (16.321 g). The water layer was separated from the upper organic layer in a separatory funnel. The organic layer was dried with MgSO4 and filtered. GC analysis of the resulting liquid showed the yield, on a mole basis from the starting lactone, was 1.1% GBL, 68.9% MBL. No oxa-Michael species were present, but higher boiling species were present. Following the calculation method of '474, product relative to starting material was 98.4%. Confirming the benefit of neutralization of after the first stage.

The alpha-methylene-gamma-butyrolactones are useful as monomers for preparing high Tg homopolymers or copolymerized by various methods for applications where their low volatility, increased polymer rigidity, and thermal stability contribute.

We claim:

1. In a two-stage, optional-one-pot process:
a) to react, in a solvent, at least one metal alkoxide base, at least one alkyl formate ester, and a lactone in a first stage to produce an alpha carbonyl lactone salt, after which any primary alcohol present is removed by distillation under reduced pressure to less than fifty mole percent of the starting moles of lactone;
b) the reaction mixture from said first stage is further reacted in a second stage with a formaldehyde source to produce an alpha-methylene lactone;
the improvement comprising: conducting said first stage by introducing a solvent mixture comprising an aromatic hydrocarbon and either a secondary alcohol of a tertiary alcohol or both, and removal of primary alcohols by distillation under reduced pressure,

2. The process of claim 1 wherein prior to said second stage, primary alcohol is removed to less than twenty mole percent of the starting moles of lactone.

3. The process of claim 1 wherein said alpha-methylene lactone product is alpha-methylene-gamma-butyrolactone.

4. The process of claim 1 wherein said product alpha-methylene lactone is produced from the group comprising gamma-butyrolactone and gamma-alkyl-gamma-butyrolactones, where the alkyl group is linear or branched and contains 1-6 carbon atoms.

5. The process of claim 1 wherein after completion of the first stage formation of alpha-formylated lactone salt, less than five mole percent of unreacted starting lactone remains.

6. The process of claim 1 wherein said alkyl formate ester is derived from a secondary alcohol.

7. The process of claim 1 wherein-said at least one said metal alkoxide base is formed from a tertiary alcohol.

8. The process of claim 1 wherein said aromatic hydrocarbon is selected from the group consisting of benzene, toluene, ethyl benzene, xylenes, and combinations thereof.

9. The process of claim 1 wherein said primary alcohol is removed from the reaction mixture by distillation at pressures below 40 torr and at temperatures less than 40° C.

10. The process of claim 9 wherein the reaction mixture after said primary alcohol removal is diluted with a dry aromatic solvent and said lactone salt collected as a filter cake then washed with at least once with a portion of the same or different dry aromatic solvent.

11. The process of claim 1 wherein said primary alcohol is removed by refluxing the reaction medium at pressures below 40 torr and at a temperature of less than 40° C. and adsorbing the alcohol(s) from the returning condensate in activated molecular sieves.

12. The process of claim 1 wherein said first stage is conducted at a temperature greater than 10° C. and less than 50° C.

13. In a two-stage, optional-one-pot process:
a) to react, in a solvent, at last one metal alkoxide base, at least one alkyl formate ester, and a lactone in a first stage to produce an alpha-carbonyl lactone salt; after which an agent for neutralization is added to neutralize residual alkoxide base and, concomitantly form a weak base;
b) the mixture from said first stage is further reacted in a second stage with a formaldehyde source to produce an alpha-methylene lactone,
the improvement comprising: conducting said first stage by introducing a solvent mixture comprising an aromatic hydrocarbon and either a secondary alcohol or tertiary alcohol or both, and neutralization of residual alkoxide base so that filtration of the alpha-carbonyl salt can be eliminated.

14. The process of 13 wherein said alpha-methylene-lactone is selected from the group consisting of alpha-methylene-gamma-butyrolactone.

15. The process of claim 13 wherein said product alpha-methylene lactone is produced from the group comprising gamma-butyrolactone and gamma-alkyl-gamma-butyrolactones, where the alkyl group is linear or branched and contains 1-6 carbon atoms.

16. The process of claim 13 wherein after completion of the first stage formation of alpha-formylated lactone salt, less than five mole percent of unreacted starting lactone remains.

17. The process of claim 13 wherein after completion of the first stage formation of alpha-formylated lactone salt, less than five mole percent of unreacted starting lactone remains relative to the starting lactone.

18. The process of claim 13 wherein said alkyl formate ester is derived from a secondary alcohol.

19. The process of claim 13 wherein at least one said metal alkoxide base is formed from a tertiary alcohol.

20. The process of claim 13 wherein an aromatic hydrocarbon solvent is selected from the group consisting of benzene, toluene, ethyl benzene, xylenes, and combinations thereof.

21. The process of claim 13 wherein the agent for neutralizing the alkoxide base is a species having a pKa for dissociation in water between 9.0 and 13.5.

22. The process of claim 13, wherein the agent for neutralizing the alkoxide base is a species having a pKa for dissociation in water between 9.5 and 12.0.

23. The process of claim 13 wherein the agent for neutralizing the alkoxide base is a trialkylammonium salt of a carboxylic acid or a mineral acid

24. The process of claim 13 wherein the agent for neutralizing the alkoxide base is an alkali bicarbonate.

25. The process of claim 13 wherein said first stage is conducted at a temperature greater than 10° C. and less than 50° C.
